(19) United States
(12) Patent Application Publication
(10) Pub. No.: US 2016/0313176 A1
(43) Pub. Date: Oct. 27, 2016

(54) USER-WEARABLE DEVICES INCLUDING UV LIGHT EXPOSURE DETECTOR WITH CALIBRATION FOR SKIN TONE

(71) Applicant: SALUTRON, INC., Fremont, CA (US)

(72) Inventor: Yong Jin Lee, Seoul (KR)

(73) Assignee: SALUTRON, INC., Fremont, CA (US)

(21) Appl. No.: 14/792,374

(22) Filed: Jul. 6, 2015

Related U.S. Application Data
(60) Provisional application No. 62/150,685, filed on Apr. 21, 2015.

(51) Int. Cl. G01J 1/42; G01J 1/02; H01L 31/101; A61B 5/0205; A61B 5/00

(52) U.S. Cl. CPC G01J 1/429; A61B 5/0205; A61B 5/443; A61B 5/6831; H01L 31/101; G01J 1/0295; A61B 5/02028

(57) ABSTRACT

A user-wearable device includes a front facing first light detector and a backside optical sensor, which faces the user's skin and includes a light source and a second light detector. The device also includes a skin tone detector and an ultraviolet (UV) exposure detector. The UV exposure detector is adapted to determine estimate(s) of a user's exposure to UV light in dependence on signal(s) produced using the first light detector, calibrate UV exposure threshold(s) in dependence on a skin tone metric produced using the skin tone detector, compare estimate(s) of a user's exposure to UV light to calibrated UV exposure threshold(s), and selectively trigger an alert in dependence on results of the comparison(s). The second light detector is also used to produce a photoplethysmography (PPG) signal from which measures heart rate (HR), heart rate variability (HRV), respiration rate (RR) or respiratory sinus arrhythmia (RSA) is/are produced.

### PRIORITY CLAIM

This application claims priority to U.S. Provisional Patent Application No. 62/150,685, filed Apr. 21, 2015, which is incorporated herein by reference.

### BACKGROUND

User-wearable devices, such as activity monitors or actigraphs, have become popular as a tool for promoting exercise and a healthy lifestyle. Such user-wearable devices can be used, for example, to measure heart rate and/or other physiological measurements. Such user-wearable devices may also measure steps taken while walking or running and/or estimate an amount of calories burned. Additionally, or alternatively, user-wearable devices can be used to monitor sleep related metrics. It would be advantageous if such devices can be used promote further beneficial lifestyle choices, such as limiting a wearer's exposure to ultraviolet (UV) radiation.

### BRIEF DESCRIPTION OF THE DRAWINGS

FIG. 1A depicts a front view of a user-wearable device, according to an embodiment.

FIG. 1B depicts a rear view of the user-wearable device of FIG. 1A, according to an embodiment.

FIG. 2 depicts a high level block diagram of electrical components of the user-wearable device introduced in FIGS. 1A and 1B, according to an embodiment.

FIG. 3 includes a plot illustrative of the radiation spectrum of sunlight during midday and a plot of an exemplary spectral response of a typical a silicon photodiode.

FIG. 4 is a block diagram that is used to provide additional details of the front facing light sensor introduced in FIG. 1A, according to an embodiment.

FIG. 5 is a block diagram that is used to provide additional details of the optical sensor introduced in FIG. 1B, according to an embodiment.

FIG. 6 is a high level flow diagram that is used to summarize methods according to various embodiments of the present technology.

### DETAILED DESCRIPTION

In the following detailed description, reference is made to the accompanying drawings that form a part hereof, and in which is shown by way of illustration specific illustrative embodiments. It is to be understood that other embodiments may be utilized and that mechanical and electrical changes may be made. The following detailed description is, therefore, not to be taken in a limiting sense. In the description that follows, like numerals or reference designators will be used to refer to like parts or elements throughout. In addition, the first digit of a reference number identifies the drawing in which the reference number first appears.

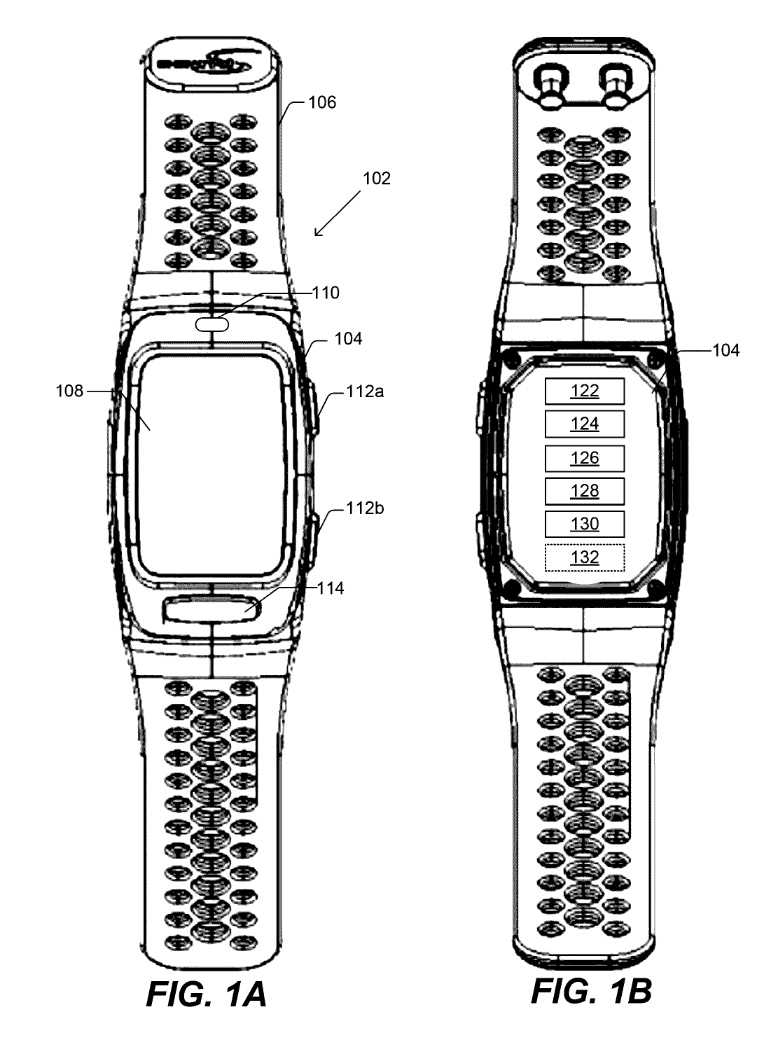

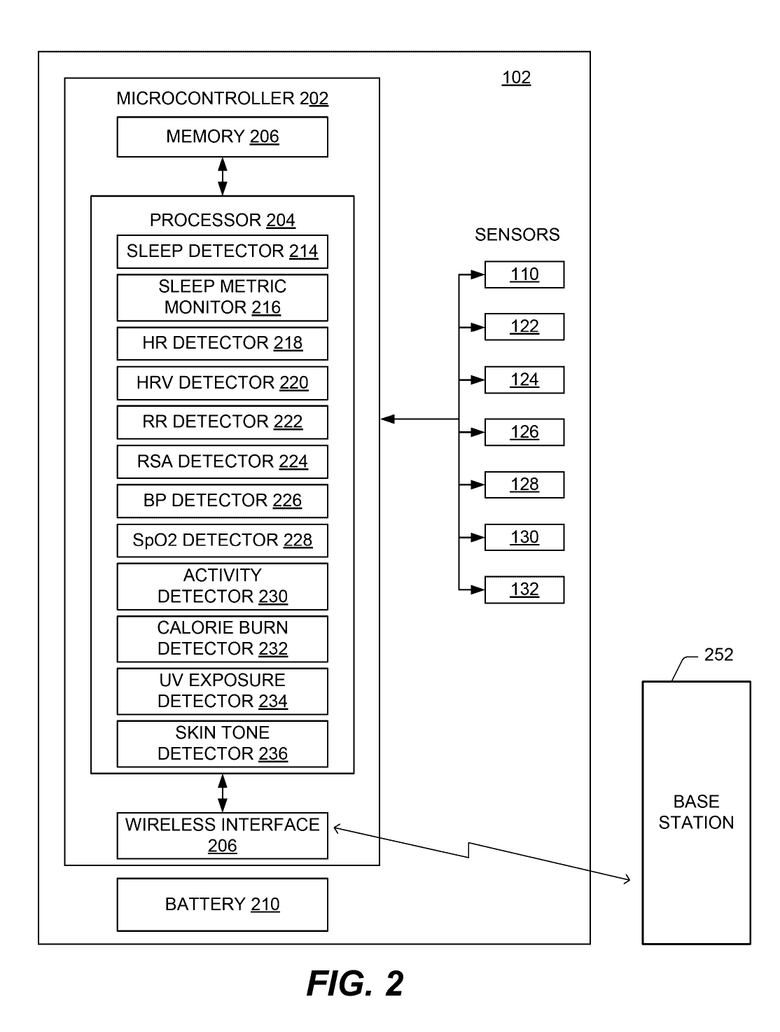

FIG. 1A depicts a front view of a user-wearable device 102, according to an embodiment of the present technology. The user-wearable device 102 can be a standalone device which gathers and processes data and displays results to a user. Alternatively, the user-wearable device 102 can wirelessly communicate with a base station (e.g., 252 in FIG. 2), which can be a mobile phone, a tablet computer, a personal data assistant (PDA), a laptop computer, a desktop computer, or some other computing device that is capable of performing wireless communication. The base station can, e.g., include a health and fitness software application and/or other applications, which can be referred to as apps. The user-wearable device 102 can upload data obtained by the device 102 to the base station, so that such data can be used by a health and fitness software application and/or other apps stored on and executed by the base station.

The user-wearable device 102 is shown as including a housing 104, which can also be referred to as a case 104. A band 106 is shown as being attached to the housing 104, wherein the band 106 can be used to strap the housing 104 to a user's wrist or arm. The housing 104 is shown as including a digital display 108, which can also be referred to simply as a display. The digital display 108 can be used to show the time, date, day of the week and/or the like. The digital display 108 can also be used to display activity and/or physiological metrics, such as, but not limited to, heart rate (HR), heart rate variability (HRV), respiratory sinus arrhythmia (RSA), calories burned, steps taken and distance walked and/or run. The digital display 108 can further be used to display sleep metrics, examples of which are discussed below. The digital display 108 can also be used to display information about a user's exposure to UV radiation (also referred to as UV light), and provide UV radiation exposure related recommendations to the user. These are just examples of the types of information that may be displayed on the digital display 108, which are not intended to be all encompassing.

The housing 104 is also shown as including an outward facing light detector 110, which can also be referred to as a light sensor 110. In accordance with an embodiment, the outward facing light detector 110 can be used to detect ambient light, during which times the light detector 110 can function as an ambient light sensor (ALS). When functioning as an ALS, the light detector 110 can be used to detect ambient light, and thus, can be useful for detecting whether it is daytime or nighttime, as well as for other purposes. In accordance with an embodiment, the same outward facing light detector 110 can also be used to detect UV light so that a user's exposure to UV radiation can be quantified and used to provide recommendations to the user. In an alternative embodiment, which is likely more costly because it requires an additional sensor, the housing includes an additional outwardly facing sensor that is dedicated to detecting UV light.

The housing 104 is further shown as including buttons 112a, 112b, which can individually be referred to as a button 112, and can collectively be referred to as the buttons 112. One of the buttons 112 can be a mode select button, while another one of the buttons 112 can be used to start and stop certain features. While the user-wearable device 102 is shown as including two buttons 112, more or less than two buttons can be included. The buttons 112 can additionally or alternatively be used for other functions. The housing 104 is further shown as including a forward facing ECG electrode 114, which is discussed below. This ECG electrode 114 can also function as an additional button.

In certain embodiments, the user-wearable device 102 can receive alerts from a base station (e.g., 252 in FIG. 2). For example, where the base station 252 is a mobile phone, the user wearable device 100 can receive alerts from the base station, which can be displayed to the user on the display 108. For a more specific example, if a mobile phone type of base station 252 is receiving an incoming phone call, then an incoming phone call alert can be displayed on the digital display 108 of the mobile device, which may or may not include the phone number and/or identity of the caller. Other types of alerts include, e.g., text message alerts, social media alerts, calendar alerts, medication reminders and exercise reminders, but are not limited thereto. The user-wearable device 102 can inform the user of a new alert by vibrating and/or emitting an audible sound.

FIG. 1B illustrates a rear-view of the housing 104 of the user-wearable device 102. Referring to FIG. 1B, the backside of the housing 104 includes an optical sensor 122, a capacitive sensor 124, a galvanic skin resistance sensor 126, an electrocardiogram (ECG) sensor 128 and a skin temperature sensor 130. It is also possible that the user-wearable device 102 includes less sensors than shown, more sensors than shown and/or alternative types of sensors. For example, the user-wearable device 102 can also include one or more type of motion sensor 132, which is shown in dotted line because it is likely completely encased with the housing 104.

In accordance with an embodiment, the optical sensor 122 includes both a light source and a light detector. The light source of the optical sensor 122 can include one or more light emitting elements, each of which can be a light emitting diode (LED), incandescent lamp or laser diode, but is not limited thereto. While infrared (IR) light sources are often employed in optical sensors, because the human eye cannot detect IR light, the light source can alternatively produce light of other wavelengths. The light detector of the optical sensor 122 can include one or more photodetectors, each of which can be a photoresistor, photodiode, phototransistor, photodarlington or avalanche photodiode, but is not limited thereto. The light source of the optical sensor 122 can be selectively driven to emit light. If an object (e.g., a user's wrist or arm) is within the sense region of the optical sensor 122, a large portion of the light emitted by the light source will be reflected off the object and will be incident on the light detector. The light detector generates a signal (e.g., a current) that is indicative of the intensity and/or phase of the light incident on the light detector, and thus, can be used to detect the presence of the user's wrist or arm. Where the signal generated by the light detector is a current signal, it can be converted to a voltage signal, if desired, using a transimpedance amplifier. The signal can be converted to a digital signal using an analog to digital converter. Additional analog and/or digital signal processing can be performed on such a signal. Regardless of whether the signal generated using the light detector is a current or voltage signal, or an analog or digital signal, such a signal can be referred to generally as a light detection signal. The optical sensor 122 may also use its light detector to operate as an ambient light detector, e.g., to detect whether or not a user is wearing the user-wearable device 102 on their wrist. When operating as an ambient light sensor, the light source of the optical sensor is inactive (i.e., does not emit light) and the light detector of the optical sensor 122 produces a signal having a magnitude that is dependent on the amount of ambient light that is incident on the optical sensor 122. It is expected that when a user is wearing the user-wearable device 102 on their wrist or arm, the light detector of the optical sensor 122 will be blocked (by the user's wrist or arm) from detecting ambient light, and thus, the signal produced the light detector will have a very low magnitude (presuming the light source of the optical sensor 122 is not emitting light).

In accordance with certain embodiments, the optical sensor 122 can be used to detect heart rate (HR), heart rate variability (HRV), respiratory rate (RR) and/or respiratory sinus arrhythmia (RSA). More specifically, the optical sensor 122 can operate as a photoplethysmography (PPG) sensor, in which case, the optical sensor 122 can also be referred to as a PPG sensor. When operating as a PPG sensor, the light source of the optical sensor 122 emits light that is reflected or backscattered by user tissue, and reflected/backscattered light is received by the light detector of the optical sensor 122. In this manner, changes in reflected light intensity are detected by the light detector, which outputs a PPG signal indicative of the changes in detected light, which are indicative of changes in blood volume. The PPG signal output by the light detector can be filtered and amplified, and can be converted to a digital signal using an analog-to-digital converter (ADC), if the PPG signal is to be analyzed in the digital domain. Each cardiac cycle in the PPG signal generally appears as a peak, thereby enabling the PPG signal to be used to detect peak-to-peak intervals, which can be used to calculate heart rate (HR) and heart rate variability (HRV). Slow oscillations in a baseline of the PPG signal are due to changes in intrathoracic pressure due to respiration. Accordingly, respiration rate (RR) can also be determined based on the PPG signal. Further, if desired, a signal indicative of respiration can be produced based on the PPG signal, by filtering and/or performing other signal processing on the PPG signal. Further, this enables the PPG signal to be used to calculate a level or magnitude of respiratory sinus arrhythmia (RSA).

In accordance with certain embodiments, the same optical sensor 112 that operates as a PPG sensor can also be used to detect a skin tone of a user, so that the detected skin tone (or a metric thereof) can be used to calibrate the user's exposure to UV radiation as detected using the outwardly facing light detector 110, or some other outwardly facing UV radiation sensor. For example, metric of skin tone, also referred to as a skin tone metric, can be used to calibrate a UV exposure threshold that is used to selectively trigger an alert that informs a user that they should reduce their UV exposure.

In accordance with certain embodiments, the optical sensor 122 includes a light source that emits light of two different wavelengths that enables the optical sensor 122 to be used as a pulse oximeter, in which case the optical sensor 122 can be used to non-invasively monitor the blood oxygen saturation (SpO2) of a user wearing the user-wearable device 102. For example, the optical sensor 122 can include one or more LED that emits red light (e.g., about 660 nm wavelength) and one or more further LED that emits infrared or near infrared light (e.g., about 940 nm wavelength), but is not limited thereto.

In accordance with an embodiment, the capacitive sensor 124 includes an electrode that functions as one plate of a capacitor, while an object (e.g., a user's wrist or arm) that is in close proximity to the capacitive sensor 124 functions as the other plate of the capacitor. The capacitive sensor 124 can indirectly measure capacitance, and thus proximity, e.g., by adjusting the frequency of an oscillator in dependence on the proximity of an object relative to the capacitive sensor 124, or by varying the level of coupling or attenuation of an AC signal in dependence on the proximity of an object relative to the capacitive sensor 124.

The galvanic skin resistance (GSR) sensor 126 senses a galvanic skin resistance. The galvanic skin resistance measurement will be relatively low when a user is wearing the user-wearable device 102 on their wrist or arm and the GSR sensor 126 is in contact with the user's skin. By contrast, the galvanic skin resistance measurement will be very high when a user is not wearing the user-wearable device 102 and the GSR sensor 126 is not in contact with the user's skin.

The ECG sensor 128 can be used to obtain an ECG signal from a user that is wearing the user-wearable device 102 on their wrist or arm (in which case the ECG sensor 128, which is an electrode, is in contact with the user's wrist or arm), and the user touches the front facing ECG electrode 114 with their other arm (e.g., with a finger of their other arm). Additionally, or alternatively, an ECG sensor can be incorporated into a chest strap that provides ECG signals to the user-wearable device 102. The skin temperature sensor 130 can be implemented, e.g., using a thermistor, and can be used to sense the temperature of a user's skin, which can be used to determine user activity and/or calories burned.

Depending upon implementation, heart rate (HR) and/or heart rate variability (HRV) can be determined based on signals obtained by the optical sensor 122 and/or the ECG sensor 128 (which can include the electrode 114). Additionally, respiration rate (RR) and/or respiratory sinus arrhythmia (RSA) level can be determined based on signals obtained by the optical sensor 122 and/or the ECG sensor 128 (which can include the electrode 114). One or more measures of HR, HRV, RR and/or RSA can be automatically determined periodically, in response to a triggering condition or event, at other specified times or based on a manual user action. For example, in a free living application, HR can be determined automatically during periods of interest, such as when a significant amount of activity is detected using the motion sensor 132.

Additional physiologic metrics can also be obtained using the sensors described herein. For example, blood pressure can be determined from the PPG and ECG signals by determining a metric of pulse wave velocity (PWV) and converting the metric of PWV to a metric of blood pressure. More specifically, a metric of PWV can be determining by determining a time from a specific feature (e.g., an R-wave) of an obtained ECG signal to a specific feature (e.g., a maximum upward slope, a maximum peak or a dicrotic notch) of a simultaneously obtained PPG signal. An equation can then be used to convert the metric of PWV to a metric of blood pressure.

In accordance with an embodiment the motion sensor 132 is an accelerometer. The accelerometer can be a three-axis accelerometer, which is also known as a three-dimensional (3D) accelerometer, but is not limited thereto. The accelerometer may provide an analog output signal representing acceleration in one or more directions. For example, the accelerometer can provide a measure of acceleration with respect to x, y and z axes. The motion sensor 132 can alternatively be a gyrometer, which provides a measure of angular velocity with respect to x, y and z axes. It is also possible that the motion sensor 132 is an inclinometer, which provides a measure of pitch, roll and yaw that correspond to rotation angles around x, y and z axes. For another example, the motion sensor 132 can include an e-Compass. It is also possible the user wear-able device 102 includes multiple different types of motion sensors, some examples of which were just described. Depending upon the type(s) of motion sensor(s) used, such a sensor(s) can be used to detect the posture of a portion of a user's body (e.g., a wrist or arm) on which the user-wearable device 102 is being worn.

FIG. 2 depicts an example block diagram of electrical components of the user-wearable device 102, according to an embodiment. Referring to FIG. 2, the user-wearable device 102 is shown as including a microcontroller 202 that includes a processor 204, memory 206 and a wireless interface 208. It is also possible that the memory 206 and wireless interface 208, or portions thereof, are external the microcontroller 202. The microcontroller 202 is shown as receiving signals from each of the aforementioned sensors 110, 122, 124, 126, 128, 130 and 132. The user-wearable device 102 is also shown as including a battery 210 that is used to power the various components of the device 102. While not specifically shown, the user-wearable device 102 can also include one or more voltage regulators that are used to step-up and or step-down the voltage provided by the battery 210 to appropriate levels to power the various components of the device 102.

The wireless interface 206 can wireless communicate with a base station (e.g., 252), which as mentioned above, can be a mobile phone, a tablet computer, a PDA, a laptop computer, a desktop computer, or some other computing device that is capable of performing wireless communication. The wireless interface 206, and more generally the user wearable device 102, can communicate with a base station 252 using various different protocols and technologies, such as, but not limited to, Bluetooth™, Wi-Fi, ZigBee or ultrawideband (UWB) communication. In accordance with an embodiment, the wireless interface 206 comprises telemetry circuitry that include a radio frequency (RF) transceiver electrically connected to an antenna (not shown), e.g., by a coaxial cable or other transmission line. Such an RF transceiver can include, e.g., any well-known circuitry for transmitting and receiving RF signals via an antenna to and from an RF transceiver of a base station 252.

Each of the aforementioned sensors 110, 122, 124, 126, 128, 130, 132 can include or have associated analog signal processing circuitry to amplify and/or filter raw signals produced by the sensors. It is also noted that analog signals produced using the aforementioned sensors 110, 122, 124, 126, 128, 130 and 132 can be converted to digital signals using one or more digital to analog converters (ADCs), as is known in the art. The analog or digital signals produced using these sensors can be subject time domain processing, or can be converted to the frequency domain (e.g., using a Fast Fourier Transform or Discrete Fourier Transform) and subject to frequency domain processing. Such time domain processing, frequency domain conversion and/or frequency domain processing can be performed by the processor 204, or by some other circuitry.

The user-wearable device 102 is shown as including various modules, including a sleep detector module 214, a sleep metric module 216, a heart rate (HR) detector module 218, a heart rate variability (HRV) detector module 220, a respiratory rate (RR) detector module 222, a respiratory sinus arrhythmia (RSA) detector module 224, a blood pressure (BP) detector module 226, an SpO2 detector module 228, an activity detector module 230, a calorie burn detector module 232, a UV exposure detector module 234, and a skin tone detector module 236. The various modules may communicate with one another, as will be explained below. Each of these modules 214, 216, 218, 220, 222, 224, 226, 228, 230, 232, 234 and 236 can be implemented using software, firmware and/or hardware. It is also possible that some of these modules are implemented using software and/or firmware, with other modules implemented using hardware. Other variations are also possible. In accordance with a specific embodiments, each of these modules 214, 216, 218, 220, 222, 224, 226, 228, 230, 232, 234 and 236 is implemented using software code that is stored in the memory 206 and is executed by the processor 204. The memory 206 is an example of a tangible computer-readable storage apparatus or memory having computer-readable software embodied thereon for programming a processor (e.g., 204) to perform a method. For example, non-volatile memory can be used. Volatile memory such as a working memory of the processor 204 can also be used. The computer-readable storage apparatus may be non-transitory and exclude a propagating signal.

The sleep detector module 214, which can also be referred to simply as the sleep detector 214, uses signals and/or data obtained from one or more of the above described sensors to determine whether a user, who is wearing the user-wearable device 102, is sleeping. For example, signals and/or data obtained using the outward facing light detector 110 and/or the motion sensor 132 can be used to determine when a user is sleeping. This is because people typically sleep in a relatively dark environment with low levels of ambient light, and typically move around less when sleeping compared to when awake. Additionally, if the user's arm posture can be detected from the motion sensor 132, then information about arm posture can also be used to detect whether or not a user is sleeping.

The sleep metric detector module 216, which can also be referred to simply as the sleep metric detector 216, uses information obtained from one or more of the above described sensors and/or other modules to quantify metrics of sleep, such as total sleep time, sleep efficiency, number of awakenings, and estimates of the length or percentage of time within different sleep states, including, for example, rapid eye movement (REM) and non-REM states. The sleep metric module 216 can, for example, use information obtained from the motion sensor 132 and/or from the HR detector 218 to distinguish between the onset of sleep, non-REM sleep, REM sleep and the user waking from sleep. One or more quality metric of the user's sleep can then be determined based on an amount of time a user spent in the different phases of sleep. Such quality metrics can be displayed on the digital display 108 and/or uploaded to a base station (e.g., 252) for further analysis.

The HR detector module 218, which can also be referred to simply as the HR detector 218, uses signals and/or data obtained from the optical sensor 122 and/or the ECG sensor 128 (which can include the electrode 114) to detect HR. For example, the optical sensor 122 can be used to obtain a PPG signal from which peak-to-peak intervals can be detected. For another example, the ECG sensor 128 (which can include the electrode 114) can be used to obtain an ECG signal, from which peak-to-peak intervals (e.g., Rwave-to-Rwave intervals) can be detected. The peak-to-peak intervals of a PPG signal or an ECG signal can also be referred to as beat-to-beat intervals, which are intervals between heart beats. Beat-to-beat intervals can be converted to HR using the equation HR=(1/beat-to-beat interval)*60. Thus, if the beat-to-beat interval=1 sec, then HR=60 beats per minute (bpm); or if the beat-to-beat interval=0.6 sec, then HR=100 bpm. The user's HR can be displayed on the digital display 108 and/or uploaded to a base station (e.g., 252) for further analysis.

The HRV detector module 220, which can also be referred to simply as the HRV detector 220, uses signals and/or data obtained from the optical sensor 122 and/or the ECG sensor 128 (which can include the electrode 114) to detect HRV. For example, in the same or a similar manner as was explained above, beat-to-beat intervals can be determined from a PPG signal obtained using the optical sensor 122 and/or from an ECG signal obtained using the ECG sensor 128 (which can include the electrode 114). HRV can be determined by calculating a measure of variance, such as, but not limited to, the standard deviation (SD), the root mean square of successive differences (RMSSD), or the standard deviation of successive differences (SDSD) of a plurality of consecutive beat-to-beat intervals. Alternatively, or additionally, obtained PPG and/or ECG signals can be converted from the time domain to the frequency domain, and HRV can be determined using well known frequency domain techniques. The user's HRV can be displayed on the digital display 108 and/or uploaded to a base station (e.g., 252) for further analysis.

The RR detector module 222, which can also be referred to simply as the RR detector 222, uses signals and/or data obtained from the optical sensor 122 to detect respiratory rate (RR). The RSA detector module 224, which can also be referred to simply as the RSA detector 224, uses signals and/or data obtained from the optical sensor 122 to detect respiratory sinus arrhythmia (RSA). In accordance with an embodiment, the RR detector 222 and/or the RSA detector 224 can communicate with the HRV detector 220 to estimate RR and/or RSA based on HRV and changes therein, as is known in the art.

The BP detector module 226, which can also be referred to simply as the BP detector 226, uses signals and/or data obtained from the optical sensor 122 and the ECG sensor 128 (which can include the electrode 114) to detect a measure of blood pressure (BP). For example, the BP detector 226 can determine a metric of pulse wave velocity (PWV) from a PPG obtained using the optical sensor 122 and an ECG signal obtained using the ECG sensor and can convert the metric of PWV to a metric of blood pressure. The metric of PWV can be determining by determining a time from a specific feature (e.g., an R-wave) of an obtained ECG signal to a specific feature (e.g., a maximum upward slope, a maximum peak or a dicrotic notch) of a simultaneously obtained PPG signal. The BP detector 226 can then be use one or more well-known equations to convert the metric of PWV to one or more metrics of blood pressure, including, but not limited to, systolic blood pressure (SBP) and diastolic blood pressure (DSP).

The SpO2 detector module 228, which can also be referred to simply as the SpO2 detector 228, uses signals and/or data obtained from the optical sensor 122 to detect blood oxygen saturation (SpO2). In order to enable the SpO2 detector 228 to detect SpO2, the optical sensor alternately emits light of two different wavelengths, typically red (e.g., about 660 nm wavelength) and infrared or near infrared (e.g., about 940 nm wavelength), which light is reflected by user tissue such that a light detector of the optical sensor 122 receives incident light that alternates between red and infrared light. As the light is reflected from tissue, some of the energy is absorbed by arterial and venous blood, tissue and the variable pulsations of arterial blood. An interleaved stream of red and infrared light is received by the light detector of the optical sensor 122. The amplitudes of the red light pulses in the light stream are differently effected by the absorption than the infrared light pulses, with the absorptions levels depending on the SpO2 level of the blood. The SpO2 detector 228 can then be use one or more well-known equations to convert relative values indicative of the amount of red and infrared light detected to values of SpO2.

The activity detector module 230, which can also be referred to simply as the activity detector 230, can determine a type and amount of activity of a user based on information such as, but not limited to, motion data obtained using the motion sensor 132, heart rate as determined by the HR detector 218, an amount of ambient light as determined using the outwardly facing light detector 110, skin temperature as determined by the skin temperature sensor 130, and time of day. The activity detector module 230 can use motion data, obtained using the motion sensor 132, to determine the number of steps that a user has taken with a specified amount of time (e.g., 24 hours), as well as to determine the distance that a user has walked and/or run within a specified amount of time. Activity metrics can be displayed on the digital display 108 and/or uploaded to a base station (e.g., 252) for further analysis.

The calorie burn detector module 232, which can also be referred to simply as the calorie burn detector 230, can determine a current calorie burn rate and an amount of calories burned over a specified amount of time based on motion data obtained using the motion sensor 132, HR as determined using the HR detector 218, and/or skin temperature as determined using the skin temperature sensor 130. A calorie burn rate and/or an amount of calories burned can be displayed on the digital display 108 and/or uploaded to a base station (e.g., 252) for further analysis.

The UV detector module 234, which can also be referred to simply as the UV detector 234, can determine estimates of a user's present exposure to UV light and/or cumulative exposure to UV light over a specified period of time (e.g., 1 hour or 24 hours, but not limited thereto). A present exposure to UV light and/or a cumulative exposure to UV light over a specified period of time can be displayed on the digital display 108 and/or uploaded to a base station (e.g., 252) for further analysis. Additionally, the UV detection module 234 can calculate a recommended maximum exposure time to UV radiation to prevent sunburns and other harmful effects of UV radiation. Further, the UV detector module 234 can selectively trigger an alert, e.g., recommending that a user reduce their exposure to UV light. Such an alert can be a textual and/or pictorial alert that is displayed on the digital display 108. Additionally, or alternatively, the alert can be an auditory alert. It is also possible that the UV detector module 234 transmits, via the wireless interface 206, data to the base station 252 that instructs or otherwise causes the base station to issue such an alert.

The skin tone detector module 236, which can also be referred to simply as the skin tone detector 236, can produce a skin tone metric indicative of a skin tone of the user wearing the user-wearable device 102. In accordance with certain embodiments, the skin tone metric produced by the skin tone detector 236 can be used to calibrate the UV detector module 234, since it is typically the case the people with a darker skin tone can tolerate more UV exposure than people with a lighter skin tone. More specifically, the UV detector module 234 can calibrate a present UV exposure threshold and/or a cumulative UV exposure threshold in dependence on the skin tone of a user, and can use the UV exposure threshold(s) to determine when to trigger alerts. For example, there can be a default present UV exposure threshold (DPUVET) and a default cumulative UV exposure threshold (DCUVET). In certain embodiments the skin tone detector can determine a skin tone value (STV) between 1 and 2, with 1 indicating a lightest skin tone and 2 indicating a darkest skin tone, and values between 1 and 2 indicating varying degrees or levels of skin tone. Such a skin tone value (STV) is an example of a skin tone metric. In accordance with a particular embodiment, a calibrated present UV exposure threshold (CPUVET) is determined using the equation: CPAVET=DPUVET*STV. Similarly, a calibrated cumulative UV exposure threshold (CCUVET) can be determined using the equation: CCAVET=DCUVET*STV. In accordance with an embodiment, the UV detector module 234 compares an estimate of present UV exposure to the calibrated present UV exposure threshold (CPUVET), and if the threshold is exceeded triggers an alert. Additionally, or alternatively, the UV detector module 234 compares an estimate of cumulative UV exposure to the calibrated cumulative UV exposure threshold (CCUVET), and if the threshold is exceeded triggers an alert.

As noted above, the optical sensor 122 on the backside of the housing 104 includes a light source and a light detector, e.g., as shown in FIG. 5 discussed below. In accordance with certain embodiments, the skin tone detector 236 uses the optical sensor 122 to determine a skin tone value (STV) based on a magnitude of reflected light, originating from the light source of the optical sensor 122, that is incident on the light detector of the optical sensor 122. More specifically, the light detector of the optical sensor 122 can produce a light detection signal indicative of reflected light incident on the light detector of the optical sensor 122. The magnitude of the light detection signal will be dependent on the distance between the target and the optical sensor and the color of the target/object off of which light reflected. In general, all other things being equal, the closer a target/object to the optical sensor 122, the greater the magnitude of the light detection signal. Further, all other things being equal, if a target/object has a white color, or another highly reflective color, the magnitude of the light detection signal will be greater than if the target has a black color, or another lowly reflective color. It can be assumed that when a person is wearing the user-wearable device 102 on their wrist, the distance between the person's wrist and the optical sensor 122 (on the backside of the housing 104) is relatively constant, and thus, that any variation in the magnitude of the light detection signal is primarily due to the color or tone of the skin of the person wearing the user-wearable device 102. Accordingly, in accordance with certain embodiments of the present technology, the skin tone detector module 236 determines the skin tone value (STV) for a user in dependence on a magnitude of a light detection signal produced using the optical sensor 122, wherein the same optical sensor 122 can also be used to obtain PPG signals for producing measures of HR, HRV, blood pressure, and or other physiological parameters. Where the skin tone value (STV) is a value between 1 and 2, with 1 indicating a lightest skin tone and 2 indicating a darkest skin tone, and values between 1 and 2 indicating varying degrees or levels of skin tone, the STV value can be inversely related to (e.g., inversely proportional to) the magnitude of the light detection signal. In other words, a light detection signal having a relatively low magnitude is indicative of a person having a dark skin tone, and thus a relatively high STV, where it is assumed that the darker the skin tone the greater the STV value. Conversely, a light detection signal having a relatively high magnitude is indicative of a person having a light skin tone, and thus a relatively low STV, where it is assumed that the lighter the skin tone the lower the STV value.

Melanin is the pigment that gives human skin its color, with dark-skinned people have more melanin in their skin than light-skinned people have. Erythema is redness of the skin caused by hyperemia of (i.e., increases of blood flow to) superficial capillaries. In accordance with certain embodiments, the skin tone detector 236 can distinguish melanin from erythema by analyzing the spectra as well as by analyzing a recorded history of reflectance. More specifically, the flushing of the skin will be characterized by the spectra of hemoglobin, and melanin will have a different (broader) spectra that erythema.

As mentioned above, the outward facing light detector 110 can be used to detect UV light so that a user's exposure to UV radiation can be quantified and used to provide recommendations to the user. The light detector 110 can include one or more photodetectors, and more specifically, each photodetector can be a photodiode that converts light that is incident on the photodiode to a current signal, which is optionally converted to a voltage signal using a transimpedance amplifier. While special photodetectors exist that are specifically adapted to detect UV radiation, such special photodetectors are typically more expensive than conventional silicon photodetectors and such special photodetectors are typically not as useful for detecting ambient visible light and/or IR light. In accordance with specific embodiments of the present technology, described below, the outward facing light detector 110, which is used to detect UV light, includes conventional silicon photodetectors and uses such photodetectors to detect UV light so that a user's exposure to UV radiation can be quantified, or more generally estimated, and used to provide recommendations to the user.

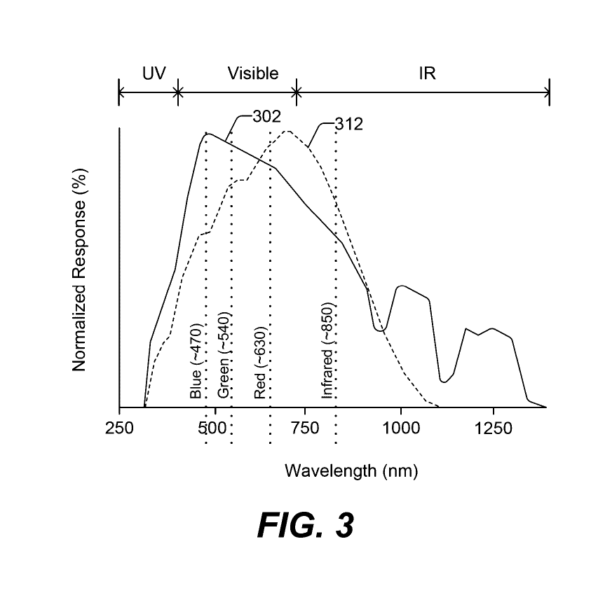

Referring now to FIG. 3, a plot 302 is illustrative of the radiation spectrum of sunlight during midday. Stated another way, the plot 302 illustrates the spectra of sunlight at midday. In actuality, the spectra of sunlight 302 will vary depending upon the time of the day, the day of the year, and the geographical latitude at which the sunlight is being measured. Sunlight includes ultraviolet (UV) light, also referred to a UV radiation, within the range of about 290 nm-400 nm, which includes both UVA radiation (320 nm-400 nm) and UVB radiation (390-320 nm). While the sun also produces UVC radiation (100-290 nm), such UVC radiation is typically completed absorbed by the ozone layer and atmosphere, and thus, does not typically reach the Earth's surface. Sunlight also includes visible light, also referred to as visible radiation, within the range of about 380 nm-740 nm. Such visible light includes, inter alia, red (R), green (G) and blue (B) light. Additionally, sunlight includes infrared (IR) light, also referred to as IR radiation, from about 740 nm-1400 nm.

Also shown in FIG. 3 is a plot 312 illustrating an exemplary spectral response of a typical conventional silicon photodiode. As can be appreciated from FIG. 3, a silicon photodiode is not optimized for measuring UV radiation. In accordance with specific embodiments of the present technology, rather than using a light detector to directly measure UV radiation, a plurality of silicon photodiodes of a light detector (e.g., 110) are used to indirectly measure UV radiation. More specifically, in accordance with specific embodiments described herein, the light detector 110 includes a plurality of silicon photodetectors (e.g., silicon photodiodes) covered by various colored filters. More specifically, the outwardly facing light detector 110 can include one or more silicon photodetectors adapted to be primarily responsive to red light and thereby produce a signal indicative of red light that is incident on the light detector 110. Such silicon photodetector(s) can be, e.g., silicon photodiode(s) covered by a red filter. Additionally, the light detector 110 can include one or more silicon photodetectors adapted to be primarily responsive to green light and thereby produce a signal indicative of green light that is incident on the light detector 110. Such silicon photodetector(s) can be, e.g., silicon photodiode(s) covered by a green filter. The light detector 110 can also include one or more silicon photodetectors adapted to be primarily responsive to blue light and thereby produce a signal indicative of blue light that is incident on the light detector 110. Such silicon photodetector(s) can be, e.g., silicon photodiodes covered by a blue filter. Additionally, the light detector 110 can include one or more silicon photodetectors adapted to be primarily responsive to infrared light and thereby produce a signal indicative of infrared light that is incident on the light detector 110. Such silicon photodetector(s) can be, e.g., silicon photodiode(s) covered by an IR filter. In other words, the light detector 110 can be an RGB and IR sensor that includes four channels, one of which produces a light detection signal (current or voltage) indicative of a magnitude of red light incident on the light detector 110, one of which produces a light detection signal indicative of a magnitude of green light incident on the light detector 110, one of which produces a light detection signal indicative of a magnitude of blue light incident on the light detector 110, and one of which produces a light detection signal indicative of a magnitude of IR light incident on the light detector 110. More generally, the light detector 110 on or adjacent the front side of the housing 104 of the user-wearable device 110 can include a plurality of photodetectors each of which is adapted to be primarily responsive to a different wavelength of visible light or infrared light and to produce a signal indicative of the wavelength of visible or infrared light to which the photodetector is primarily responsive. In such embodiments, the UV exposure detector 234 can be adapted to determine estimates of a user's exposure to UV light in dependence on the signals indicative of the visible or infrared light produced using the light detector 110.

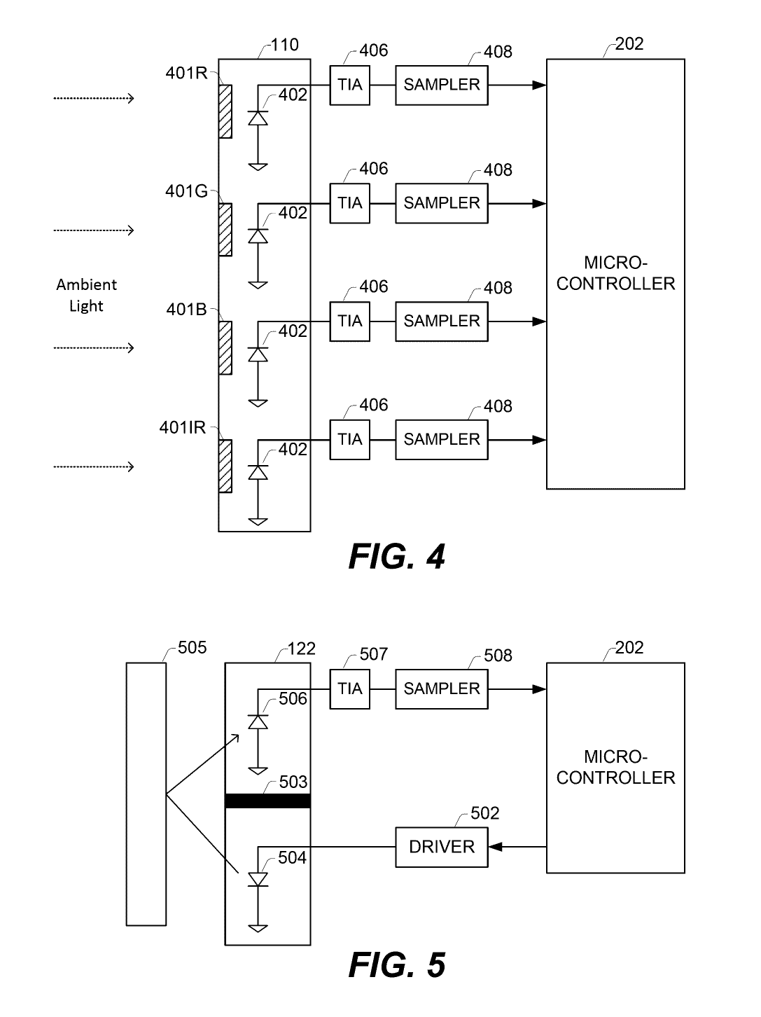

FIG. 4 illustrates exemplary details of the front facing light detector 110, which is on or adjacent the front side of the housing 104 and is adapted to produce one or more light detections signals indicative of ambient light that is incident on the light detector 110. Referring to FIG. 4, the light detector 110, which can also be referred as the light sensor 110, is shown as including a photodetector 402 covered by a red filter 401R, a photodetector 402 covered by a green filter 401G, a photodetector 402 covered by a blue filter 401B and a photodetector 402 covered by an IR filter 401IR. In accordance with specific embodiments, each of the photodetectors 402 comprises a silicon photodiode, an exemplary spectral response for which is shown in FIG. 3. It is also possible that the light detector 110, which can also be referred to as the light sensor 110, includes multiple photodetectors (e.g., multiple silicon photodiodes) covered by a red filter, multiple photodetectors (e.g., multiple silicon photodiodes) covered by a green filter, multiple photodetectors (e.g., multiple silicon photodiodes) covered by a blue filter, and multiple photodetectors (e.g., multiple silicon photodiodes) covered by an IR filter. There can additionally be one or more photodetectors (e.g., multiple silicon photodiodes) that is/are not covered by any color filter. Where the signals generated by the photodetectors 402 are current signals, transimpedance amplifiers (TIAs) 406 can be used to convert the current signals to voltage signals. Further analog circuitry, not specifically shown in FIG. 4, can be used to perform analog signal filtering, and/or analog signal amplification of signals produced by the photodetectors of the light detector 110, which can also be referred to as the light sensor 110. Still referring to FIG. 4, samplers 408 are shown as sampling the light detection signals produced using the photodetectors 402 of the light detector 408. The samplers 408 can alternatively be implemented within and by the microcontroller 202.

In accordance with an embodiment, the R, G, B and IR characteristics of sunlight at different times of the day are measured (and optionally at different days of the year and/or at different geographical latitudes), using the light detector 110 (or a replica thereof), and UV characteristics are also measured using a UV light sensor (not shown) that is specifically adapted to measure light within the UV range of wavelengths. Based on such measurements, an algorithm and/or lookup-table is produced that correlates measurements of R, G, B and IR characteristics with measurements of UV light. Such an algorithm and/or lookup-table is stored, e.g., in the memory 206, and used by the UV exposure detector 234 to indirectly detect UV light in dependence on measurements of R, G, B and IR light produced using the light detector 110. In other words, silicon photodetectors, which are not optimized for measuring UV radiation, are used to indirectly measure UV radiation. In certain embodiments, measurements of R, G, B and IR light are used to indirectly measure UV radiation. Measurements of additional and/or different colors or wavelengths of light can alternatively be used to indirectly measure UV radiation. It is also possible that less than or more than four different wavelengths or colors of light can be used to indirectly measure UV radiation. For example, measurements of only three of four (of R, G, B and IR light) can be used to indirectly measure UV radiation. Other variations are also possible and are within embodiments of the present technology.

FIG. 5 illustrates exemplary details of the optical sensor 122 that is on or adjacent the back side of the housing 104. Referring to FIG. 5, the optical sensor 122 is shown as including a light source 504 and a light detector 506. The light source 504, as mentioned above, can include one or more LED, incandescent lamp or laser diode, but is not limited thereto. The light detector 506 can include one or more one or more photoresistor, photodiode, phototransistor, photodarlington or avalanche photodiode, but is not limited thereto. A driver 502, whose timing is controlled by the microcontroller 202, drives the light source 504 to emit light at a low frequency, a high frequency, or continually. The light detector 506 generates a signal (e.g., a current) that is indicative of the intensity and/or phase of the light incident on the light detector 506. Where the signal generated by the light detector 506 is a current signal, a transimpedance amplifier (TIA) 507 can be used to convert the current signal to a voltage signal. Further analog circuitry, not specifically shown in FIG. 5, can be used to perform analog signal filtering, and/or analog signal amplification of a signal produced by the one or more light detecting elements of the light detector 506. Still referring to FIG. 5, a sampler 508 is shown as sampling the light detection signal produced using the light detector 506. The sampler 508 can alternatively be implemented within and by the microcontroller 202. Element 503 is an opaque barrier that optically isolates the light source 504 from the light detector. As can be appreciated from FIG. 5, the light detector 506 detects light emitted by the light source 504 that reflects off of an object 505 and is incident on the light detector 506. The object 505 can be, e.g., the wrist of a user or some other portion of the user's body.

As mentioned above, the optical sensor 122 can be used to produce a PPG signal, wherein the peak-to-peak magnitudes of the PPG signal, or more generally, of the light detection signal produced by the light detector 506, are indicative of changes in blood volume. An average magnitude, or baseline, of the PPG signal is dependent on the color of the user's skin tone, because light skin is more reflective than dark skin, and dark skin is more absorptive that light skin. In accordance with certain embodiments, the user's skin tone, or more generally a metric indicative of the user's skin tone, is determined by determining an average magnitude of a PPG signal produced using the optical sensor 122. As noted above, this metric of skin tone can be used to calibrate one or more UV exposure thresholds. More specifically, in certain embodiments described above, the skin tone detector 236 can produce a skin tone value (STV) based on a light detection signal produced using the optical sensor 122, and the skin tone value (STV) can be used to produce a calibrated present UV exposure threshold (CPUVET) and/or a calibrated cumulative UV exposure threshold (CCUVET), which threshold(s) are used to trigger alerts, or the like.

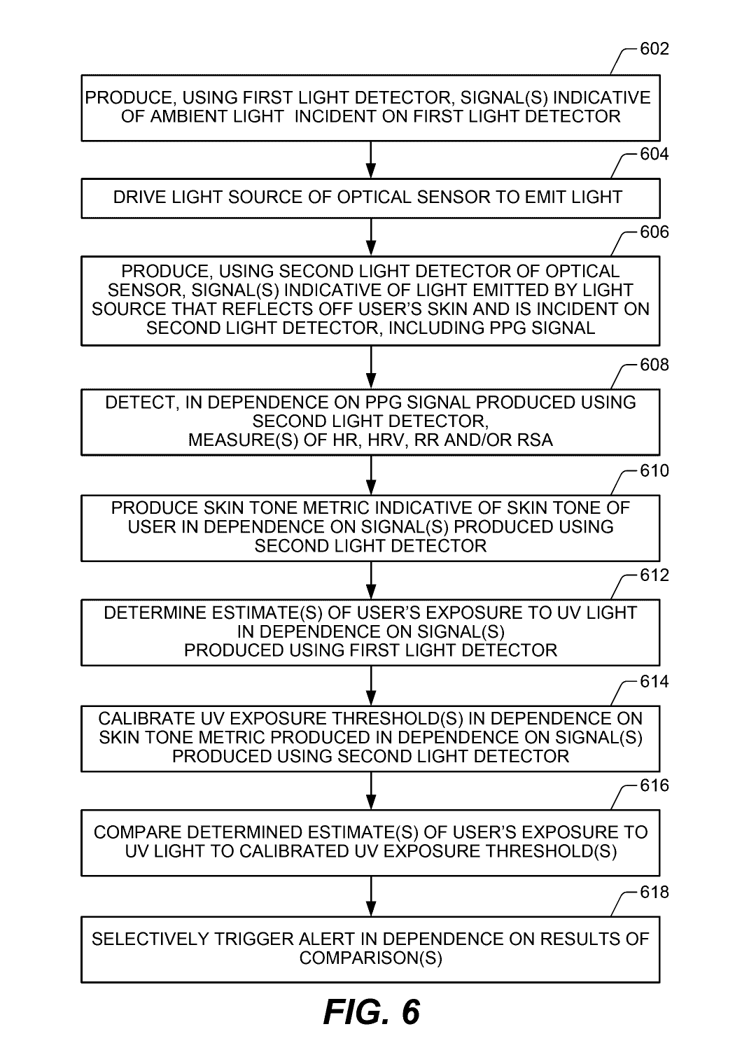

FIG. 6 will now be used to summarize methods according to various embodiments of the present technology. Such methods are for use with a user-wearable device, such as the device 102 described above with reference to FIGS. 1A-5, but are not limited thereto. More specifically, such a user-wearable device can include a housing having a front side and a back side, and a band that straps the housing to a user's wrist or other appendage such that the back side of the housing is positioned against a user's skin. Additionally, the user-wearable device includes a first light detector on or adjacent the front side of the housing, and an optical sensor on or adjacent the back side of the housing, wherein the optical sensor includes a light source and a second light detector. The term “second” here is used to distinguish from the “first” light detector on or adjacent the front side of the housing, and does not imply that the optical sensor (on or adjacent the back side of the housing) must include at least two light detectors.

Referring to FIG. 6, step 602 involves producing, using a first light detector, one or more signals indicative of ambient light that is incident on the first light detector, wherein the first light detector (e.g., 110) is on or adjacent a front side of a housing of a user-wearable device. Step 604 involves driving a light source of an optical sensor (e.g., 122) to emit light, wherein the optical sensor is on or adjacent the back side of the housing of the user-wearable device and also includes a second light detector. Step 606 involves producing, using the second light detector of the optical sensor, one or more signals indicative of light emitted by the light source that reflects off of a user's skin and is incident on the second light detector, wherein at least one of the one or more signals produced using the second light detector comprises a photoplethysmography (PPG) signal indicative of changes in arterial blood volume. Step 608 involves detecting, in in dependence on a PPG signal produced using the second light detector, one or more measure of heart rate (HR), heart rate variability (HRV), respiration rate (RR) or respiratory sinus arrhythmia (RSA). Step 610 involves producing a skin tone metric indicative of a skin tone of a user in dependence on at least one of the one or more signals produced using the second light detector. Step 612 involves determining at least one estimate the user's exposure to UV light in dependence on at least one of the one or more signals produced using the first light detector. Step 614 involves calibrating at least one UV exposure threshold in dependence on the skin tone metric, indicative of the skin tone of the user, which is produced in dependence on at least one of the one or more signals produced using the second light detector. Step 616 involves comparing at least one estimate of the user's exposure to UV light to at least one calibrated UV exposure threshold. Step 618 involves selectively triggering an alert in dependence on results of the comparison(s) performed at step 616. It should be understood that the order of at least some of the above steps can be rearranged while still being within the scope of an embodiment.

In accordance with certain embodiments, the first light detector includes a plurality of photodetectors each of which is adapted to be primarily responsive to a different wavelength of visible light or infrared light and thereby produce a signal indicative of the wavelength of visible or infrared light to which the photodetector is primarily responsive. In such an embodiment, step 602 involves producing multiples signals each of which is indicative of a different wavelength of visible light or infrared light that is incident on the first light detector, and step 612 involves indirectly determining estimates of the user's exposure to UV light based on the signals produced at step 602. For a more specific example, step 602 can include producing a signal indicative of red light incident on the light detector, producing a signal indicative of blue light incident on the light detector, producing a signal indicative of green light incident on the light detector, and producing a signal indicative of infrared light incident on the light detector; and step 612 can include determining at least one estimate the user's exposure to UV light in dependence on the signals indicative of red, green, blue and infrared light that are incidence on the first light detector.

In accordance with an embodiment, step 612 involves determining an estimate of the user's present exposure to UV light, step 614 involves calibrating a present UV exposure threshold (in dependence on the skin tone metric, indicative of the skin tone of the user, which is produced in dependence on at least one of the one or more signals produced using the second light detector), step 616 involves comparing the estimate of the user's present exposure to UV light to the calibrated present UV exposure threshold, and step 618 involves selectively triggering an alert based on the comparison performed at step 616. Alternatively, or additionally, step 612 involves determining an estimate of the user's cumulative exposure to UV light, step 614 involves calibrating a cumulative UV exposure threshold (in dependence on the skin tone metric, indicative of the skin tone of the user, which is produced in dependence on at least one of the one or more signals produced using the second light detector), step 616 involves comparing the estimate of the user's cumulative exposure to UV light to the calibrated cumulative UV exposure threshold, and step 618 involves selectively triggering an alert based on the comparison(s) performed at step 616. As noted above, the alert, which may recommend that a user reduce their exposure to UV light, can be a textual and/or pictorial alert that is displayed on a digital display (e.g., 108) of the user-wearable device. Additionally, or alternatively, the alert can be an auditory alert. It is also possible that the alert be issued by a base station in wireless communication with the user-wearable device.

Certain embodiments of the present technology described herein are directed to a user-wearable device comprising a housing, a band, a first light detector, an optical sensor, one or more physiologic parameter detectors, a skin tone detector and an ultraviolet (UV) exposure detector. In certain embodiments, the housing, which has a front side and a back side, can be strapped by the band to a user's wrist or other appendage such that the back side of the housing is positioned against a user's skin. The first light detector is on or adjacent the front side of the housing and is adapted to produce one or more signals indicative of ambient light that is incident on the first light detector. The optical sensor, which is on or adjacent the back side of the housing, includes a light source that emits light in response to being driven and a second light detector adapted to produce one or more signals indicative of light emitted by the light source that reflects off of a user's skin and is incident on the second light detector. The term “second” here is used to distinguish from the “first” light detector on or adjacent the front side of the housing, and does not imply that the optical sensor (on or adjacent the back side of the housing) must include at least two light detectors. At least one of the one or more signals produced using the second light detector comprises a photoplethysmography (PPG) indicative of changes in arterial blood volume. The one or more physiologic parameter detectors is/are adapted to detect one or more measure of heart rate (HR), heart rate variability (HRV), respiration rate (RR) or respiratory sinus arrhythmia (RSA) in dependence on a PPG signal produced using the second light detector. For example, the one or more physiologic parameter detectors can include an HR detector adapted to detect HR in dependence on a PPG signal produced using the second light detector. Additionally, or alternatively, the one or more physiologic parameter detectors also include an HRV detector adapted to detect HRV in dependence on a PPG signal produced using the second light detector.

In accordance with certain embodiments, the skin tone detector is adapted to produce a skin tone metric indicative of a skin tone of a user in dependence on at least one of the one or more signals produced using the second light detector. The ultraviolet (UV) exposure detector is adapted to determine at least one estimate a user's exposure to UV light in dependence on at least one of the one or more signals produced using the first light detector. The UV exposure detector is also adapted to calibrate at least one UV exposure threshold in dependence on the skin tone metric produced using the skin tone detector in dependence on at least one of the one or more signals produced using the second light detector. Further, the UV exposure threshold is adapted to compare at least one determined estimate of a user's exposure to UV light to at least one calibrated UV exposure threshold, and to selectively trigger an alert in dependence on results of the comparison(s) of the determined estimate(s) of the user's exposure to UV light to the calibrated UV exposure threshold(s).

In accordance with certain embodiments, the at least one estimate of a user's exposure to UV light comprises an estimate of the user's present exposure to UV light. In such embodiments, the at least one UV exposure threshold comprises a present UV exposure threshold. Alternatively, or additionally, the at least one estimate of a user's exposure to UV light comprises an estimate of the user's cumulative exposure to UV light, and the at least one UV exposure threshold comprises a cumulative UV exposure threshold.

In accordance with certain embodiments, the first light detector is adapted to produce signals indicative of red, green, blue and infrared light that are incidence on the first light detector. In such embodiment, the UV exposure detector is adapted to produce estimates of an amount of UV light that is incident on the first light detector in dependence on the signals indicative of red, green, blue and infrared light that are incidence on the first light detector. Further, the UV exposure detector is adapted to use the estimates of the amount of UV light that is incident on the first light detector to determine estimate(s) of the user's present exposure and/or cumulative exposure to UV light.

In accordance with certain embodiments, the first light detector comprises a plurality of silicon photodetectors including one or more silicon photodetectors adapted to be primarily responsive to red light and thereby produce a signal indicative of red light that is incident on the first light detector, one or more silicon photodetectors adapted to be primarily responsive to green light and thereby produce a signal indicative of green light that is incident on the first light detector, one or more silicon photodetectors adapted to be primarily responsive to blue light and thereby produce a signal indicative of blue light that is incident on the first light detector, and one or more silicon photodetectors adapted to be primarily responsive to infrared light and thereby produce a signal indicative of infrared light that is incident on the first light detector. The one or more photodetectors adapted to be primarily responsive to red light is/are covered by a red filter. The one or more photodetectors adapted to be primarily responsive to green light is/are covered by a green filter. The one or more photodetectors adapted to be primarily responsive to blue light is/are covered by a blue filter. The one or more photodetectors adapted to be primarily responsive to infrared light is/are covered by an infrared filter. Each photodetector can be, e.g., a silicon photodiode covered by a respective colored filter.

In accordance with certain embodiments, the skin tone detector is adapted to distinguish between melanin and erythema so that the produced skin tone metric is primarily indicative of melanin.

Referring briefly back to FIGS. 1A and 1B, the user-wearable device 102 was generally shown and described as being a wrist-wearable device that can be strapped to a user's wrist, or another portion of a user's arm. However, embodiments described herein should not be limited to use with wrist-wearable devices. For example, embodiments described herein can also be used with chest-wearable, head-wearable or leg-wearable devices, but are not limited thereto. In other words, the user-wearable devices described herein are not intended to be limited to the form factors shown in the FIGS. and described above. More generally, embodiments of the present technology described herein can be used with most any user-wearable device having a housing having a backside adapted to be worn against a user's skin, and a front side that is adapted to face outward and be exposed to ambient light.

The foregoing detailed description of the technology herein has been presented for purposes of illustration and description. It is not intended to be exhaustive or to limit the technology to the precise form disclosed. Many modifications and variations are possible in light of the above teaching. The described embodiments were chosen to best explain the principles of the technology and its practical application to thereby enable others skilled in the art to best utilize the technology in various embodiments and with various modifications as are suited to the particular use contemplated. It is intended that the scope of the technology be defined by the claims appended hereto. While various embodiments have been described above, it should be understood that they have been presented by way of example, and not limitation. It will be apparent to persons skilled in the relevant art that various changes in form and detail can be made therein without departing from the spirit and scope of the technology. The breadth and scope of the present technology should not be limited by any of the above-described exemplary embodiments, but should be defined only in accordance with the following claims and their equivalents.

What is claimed is:

1. A user-wearable device, comprising:
a housing having a front side and a back side;
a band that straps the housing to a portion of a user's body such that the back side of the housing is positioned against a user's skin;
a first light detector on or adjacent the front side of the housing and adapted to produce one or more signals indicative of ambient light that is incident on the first light detector;
an optical sensor on or adjacent the back side of the housing, including a light source that emits light in response to being driven and a second light detector adapted to produce one or more signals indicative of light emitted by the light source that reflects off of a user's skin and is incident on the second light detector, wherein at least one of the one or more signals produced using second light detector comprises a photoplethysmography (PPG) signal indicative of changes in arterial blood volume;
one or more physiologic parameter detectors adapted to detect one or more measures of heart rate (HR), heart rate variability (HRV), respiration rate (RR) or respiratory sinus arrhythmia (RSA) in dependence on a said PPG signal produced using the second light detector;
a skin tone detector adapted to produce a skin tone metric indicative of a skin tone of a user in dependence on at least one of the one or more signals produced using the second light detector; and
an ultraviolet (UV) exposure detector adapted to
determine at least one estimate a user's exposure to UV light in dependence on at least one of the one or more signals produced using the first light detector;
calibrate at least one UV exposure threshold in dependence on the skin tone metric produced using the skin tone detector in dependence on at least one of the one or more signals produced using second light detector;
compare at least one said determined estimate of a user's exposure to UV light to at least one said calibrated UV exposure threshold; and
selectively trigger an alert in dependence on results of the comparison(s) of at least one said determined estimate of the user's exposure to UV light to at least one said calibrated UV exposure threshold.

2. The user-wearable device of claim 1, wherein:
the at least one estimate of a user's exposure to UV light comprises an estimate of a user's present exposure to UV light; and
the at least one UV exposure threshold comprises a present UV exposure threshold.

3. The user-wearable device of claim 1, wherein:
the at least one estimate of a user's exposure to UV light comprises an estimate of a user's cumulative exposure to UV light; and
the at least one UV exposure threshold comprises a cumulative UV exposure threshold.

4. The user-wearable device of claim 1, wherein:
the at least one estimate of a user's exposure to UV light comprises an estimate of a user's present exposure to UV light and an estimate of a user's cumulative exposure to UV light; and
the at least one UV exposure threshold comprises a present UV exposure threshold and a cumulative UV exposure threshold.

5. The user-wearable device of claim 1, wherein:
the first light detector is adapted to produce signals indicative of red, green, blue and infrared light that are incidence on the first light detector;
the UV exposure detector is adapted to produce estimates of an amount of UV light that is incident on the first light detector in dependence on the signals indicative of red, green, blue and infrared light that are incidence on the first light detector; and
the UV exposure detector is adapted to use the estimates of the amount of UV light that is incident on the first light detector to determine the at least one estimate of a user's exposure to UV light.

6. The user-wearable device of claim 5, wherein the first light detector comprises a plurality of silicon photodetectors including:
one or more silicon photodetectors adapted to be primarily responsive to red light and to produce a signal indicative of red light that is incident on the first light detector;
one or more silicon photodetectors adapted to be primarily responsive to green light and to produce a signal indicative of green light that is incident on the first light detector;
one or more silicon photodetectors adapted to be primarily responsive to blue light and to produce a signal indicative of blue light that is incident on the first light detector; and
one or more silicon photodetectors adapted to be primarily responsive to infrared light and to produce a signal indicative of infrared light that is incident on the first light detector.

7. The user-wearable device of claim 6, wherein:
the one or more silicon photodetectors adapted to be primarily responsive to red light is/are one or more silicon photodiodes covered by a red filter;
the one or more silicon photodetectors adapted to be primarily responsive to green light is/are one or more silicon photodiodes covered by a green filter;
the one or more silicon photodetectors adapted to be primarily responsive to blue light is/are one or more silicon photodiodes covered by a blue filter; and
the one or more silicon photodetectors adapted to be primarily responsive to infrared light is/are one or more silicon photodiodes covered by an infrared filter.

8. The user-wearable device of claim 1, wherein the skin tone detector is adapted to distinguish between melanin and erythema so that the produced skin tone metric is primarily indicative of melanin.

9. The user-wearable device of claim 1, wherein the one or more physiologic parameter detectors include a heart rate (HR) detector adapted to detect HR in dependence on a said PPG signal produced using second light detector.

10. The user-wearable device of claim 9, wherein the one or more physiologic parameter detectors also include a heart rate variability (HRV) detector adapted to detect HRV in dependence on a said PPG signal produced using second light detector.

11. A method for use with a user-wearable device including
a housing having a front side and a back side;
a band that straps the housing to a portion of a user's body such that the back side of the housing is positioned against a user's skin;
a first light detector on or adjacent the front side of the housing; and
an optical sensor on or adjacent the back side of the housing and including a light source and a second light detector;
the method comprising:
(a) producing, using the first light detector, one or more signals indicative of ambient light that is incident on the first light detector;
(b) driving the light source of the optical sensor to emit light;
(c) producing, using the second light detector of the optical sensor, one or more signals indicative of light emitted by the light source that reflects off of a user's skin and is incident on the second light detector, wherein at least one of the one or more signals produced using second light detector comprises a photoplethysmography (PPG) indicative of changes in arterial blood volume;
(d) detecting, in dependence on a said PPG signal produced using second light detector, one or more measures of heart rate (HR), heart rate variability (HRV), respiration rate (RR) or respiratory sinus arrhythmia (RSA);
(e) producing a skin tone metric indicative of a skin tone of a user in dependence on at least one of the one or more signals produced using second light detector; and
(f) determining at least one estimate a user's exposure to UV light in dependence on at least one of the one or more signals produced using first light detector;
(g) calibrating at least one UV exposure threshold in dependence on the skin tone metric, indicative of the skin tone of the user, which is produced in dependence on at least one of the one or more signals produced using second light detector;
(h) comparing at least one said determined estimate of the user's exposure to UV light to at least one said calibrated UV exposure threshold; and
(i) selectively triggering an alert in dependence on results of the comparing at least one said determined estimate of the user's exposure to UV light to at least one said calibrated UV exposure threshold.

12. The method of claim 11, wherein:
step (f) comprises determining an estimate of the user's present exposure to UV light; and
step (g) comprises calibrating a present UV exposure threshold in dependence on the skin tone metric, indicative of the skin tone of the user, which is produced in dependence on at least one of the one or more signals produced using second light detector.

13. The method of claim 11, wherein:
step (f) comprises determining an estimate of the user's cumulative exposure to UV light; and
step (g) comprises calibrating a cumulative UV exposure threshold in dependence on the skin tone metric, indicative of the skin tone of the user, which is produced in dependence on at least one of the one or more signals produced using second light detector.

14. The method of claim 11, wherein:
step (f) comprises determining an estimate of the user's present exposure to UV light and an estimate of the user's cumulative exposure to UV light; and
step (g) comprises calibrating a present UV exposure threshold and a cumulative UV exposure threshold in dependence on the skin tone metric, indicative of the skin tone of the user, which is produced in dependence on at least one of the one or more signals produced using second light detector.

15. The method of claim 11, wherein:
step (a) includes producing
a signal indicative of red light incident on the first light detector,
a signal indicative of blue light incident on the first light detector,
a signal indicative of green light incident on the first light detector, and
a signal indicative of infrared light incident on the first light detector,
step (f) includes determining the at least one estimate the user's exposure to UV light in dependence on the signals indicative of red, green, blue and infrared light that are incidence on the first light detector.

16. The method of claim 15, wherein the first light detector comprises a plurality of silicon photodetectors and step (a) includes:
using one or more silicon photodetectors adapted to be primarily responsive to red light to produce the signal indicative of red light that is incident on the first light detector;
using one or more silicon photodetectors adapted to be primarily responsive to green light to produce the signal indicative of green light that is incident on the first light detector;
using one or more silicon photodetectors adapted to be primarily responsive to blue light to produce the signal indicative of blue light that is incident on the first light detector; and
using one or more silicon photodetectors adapted to be primarily responsive to infrared light to produce the signal indicative of infrared light that is incident on the first light detector.

17. The method of claim 16, wherein:
the one or more silicon photodetectors adapted to be primarily responsive to red light is/are one or more silicon photodiodes covered by a red filter;
the one or more silicon photodetectors adapted to be primarily responsive to green light is/are one or more silicon photodiodes covered by a green filter;
the one or more silicon photodetectors adapted to be primarily responsive to blue light is/are one or more silicon photodiodes covered by a blue filter; and
the one or more silicon photodetectors adapted to be primarily responsive to infrared light is/are one or more silicon photodiodes covered by an infrared filter.

18. The method of claim 11, wherein step (e) includes distinguishing between melanin and erythema so that the produced skin tone metric is primarily indicative of melanin.

19. The method of claim 11, wherein step (d) includes detecting heart rate (HR) in dependence on a said PPG signal produced using second light detector.

20. The method of claim 19, wherein step (d) also includes detecting heart rate variability (HRV) in dependence on a said PPG signal produced using second light detector.

21. A user-wearable device, comprising:
a housing having a front side and a back side;
a band that straps the housing to a portion of user's body such that the back side of the housing is positioned against a user's skin;
a first light detector on or adjacent the front side of the housing and including a plurality of silicon photodetectors, each of which is adapted to be primarily responsive to a different wavelength of visible or infrared light and to produce a signal indicative of the wavelength of visible or infrared light to which the photodetector is primarily responsive; and
an ultraviolet (UV) exposure detector adapted to determine at least one estimate a user's exposure to UV light in dependence on the signals indicative of the wavelengths of visible or infrared light produced using first light detector.

22. The user-wearable device of claim 21, further comprising:
an optical sensor on or adjacent the back side of the housing, including a light source that emits light in response to being driven and a second light detector adapted to produce one or more signals indicative of light emitted by the light source that reflects off of a user's skin and is incident on the second light detector, wherein at least one of the one or more signals produced using second light detector comprises a photoplethysmography (PPG) indicative of changes in arterial blood volume;
one or more physiologic parameter detectors adapted to detect one or more measures of heart rate (HR), heart rate variability (HRV), respiration rate (RR) or respiratory sinus arrhythmia (RSA) in dependence on a said PPG signal produced using second light detector; and
a skin tone detector adapted to produce a skin tone metric indicative of a skin tone of a user in dependence on at least one of the one or more signals produced using second light detector;
wherein the UV exposure detector is also adapted to
calibrate at least one UV exposure threshold in dependence on the skin tone metric produced using skin tone detector in dependence on at least one of the one or more signals produced using second light detector;
compare at least one said determined estimate of a user's exposure to UV light to at least one said calibrated UV exposure threshold; and
selectively trigger an alert in dependence on results of the comparison(s) of the at least one said determined estimate of the user's exposure to UV light to the at least one said calibrated UV exposure threshold(s).
